(19) United States
(12) Patent Application Publication
(10) Pub. No.: US 2004/0028757 A1
(43) Pub. Date: Feb. 12, 2004

(54) Composition, in particular cosmetic, comprising dhea and/ or precursor or derivative of thereof, combined with at least a no-synthase inhibitor

(76) Inventors: Marie-Madeleine Cals-Grierson, Meudon (FR); Roland Roguet, Paris (FR); Guy Courchay, Sucy-en-Brie (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/332,771

(22) PCT Filed: Jun. 8, 2001
(86) PCT No.: PCT/FR01/01791

(30) Foreign Application Priority Data
Jul. 13, 2000 (FR) 00/09231

(51) Int. Cl. A61K 35/78

(52) U.S. Cl. 424/757; 424/773; 514/26; 424/752; 424/769; 424/766

(57) ABSTRACT

The invention relates to a composition intended for administration by the oral or topical route and comprising, in a physiologically acceptable medium, DHEA and/or a chemical or biological precursor or derivative of the latter, in combination with at least one NO-synthase inhibitor.

This composition can be used for cosmetic purposes, for preventing or treating irritation of the skin and/or sensitive skin and/or for signs of cutaneous aging. In an alternative form, it can be used for treating the scalp and/or hair or as reducing composition.

The NO-synthase inhibitor can be chosen in particular from: lipochroman-6, a grape extract, an olive tree extract, a Gingko biloba extract or epicatechin.

The invention relates to a composition comprising, in a physiologically acceptable medium, DHEA and/or a chemical or biological precursor or derivative of the latter, in combination with at least one NO-synthase inhibitor, and to its uses.

The term “NO-synthase” covers a family of enzymes which provide for the enzymatic catalysis of L-arginine to citrulline, during which catalysis a gas mediator with multiple functions, nitrogen monoxide or NO, is produced. Because of its electronic hyperactivity, related to the presence of an additional electron in its structure, NO can lead to damage to, indeed even destruction of, the cells and is, on this account, involved in particular in the intrinsic and/or extrinsic aging of the skin.

More generally, NO is a multifunctional signal molecule active in a great variety of systems and tissues of the body. In particular, it is well accepted that NO plays a dominating role in the skin. This is because NO can be synthesized by all the varieties of cells constituting the skin and, on this account, is involved in many complex regulating processes, such as the regulation of cell differentiation and/or proliferation, of vasodilation, of melanogenesis or of the response to environmental variations (homeostasis).

It is also involved in cutaneous inflammatory and immunological processes. This is because it is commonly accepted that NO plays a role in contact hypersensitivity reactions, in cutaneous allergic manifestations or in the immune response of the skin. Likewise, in addition to its direct pro-inflammatory role, it is the mediator between neuropeptides, such as substance P and/or calcitonin gene related peptide (CGRP), in cutaneous neurogenic inflammatory processes, resulting in its involvement in “sensitive” skin phenomena.

The involvement of NO in vasodilation means that it is associated with cutaneous erythema, particularly erythema induced by ultraviolet radiation. NO is also recognized as an intermediate in melanogenesis induced by ultraviolet radiation of B type (UV-B). Finally, NO appears to be involved in the control of sweating and in that of lipolysis (inhibiting effect) or in hair loss.

The advantage which exists in having available NO-synthase inhibitors is therefore understood. In this respect, numerous inhibitors have already been provided in the prior art. Mention may be made more particularly of N^{G}-monomethyl-L-arginine (NMMA), the methyl ester of N^{G}-nitro-L-arginine (NAME), N^{G}-nitro-L-arginine (NNA), N^{G}-amino-L-arginine (NAA), N^{G},N^{G}-dimethylarginine (the asymmetric dimethylarginine, known as ADMA), diphenyleneiodonium chloride, 2-(4-carboxyphenyl)-4,4,5,5-tetramethylimidazoline-1-oxy-3-oxide, 7-nitroindazole, N(5)-(1-iminoethyl)-L-ornithine, aminoguanidine, canavanine and ebselen.

More recently, other NO-synthase inhibitors more appropriate for a cosmetic application have been disclosed. They are in particular lipochroman-6 (FR 00/05520), a grape extract (FR 00/05521), an olive tree extract (FR 00/05522), a Gingko biloba extract (FR 00/05523) or epicatechin (FR 00/05524).

However, the need remains to strengthen the effectiveness of these compounds in the prevention and/or treatment of certain disorders, in particular cutaneous disorders.

In point of fact, it appeared to the Applicant Company that DHEA and/or its chemical or biological precursors or derivatives might make it possible to improve the effectiveness of compositions comprising NO-synthase inhibitors.

DHEA, or dehydroepiandrosterone, is a natural steroid produced essentially by the advenocortical glands. It is known for its ability to promote keratinization of the epidermis (JP-07 196 467) or in the treatment of dry skin, because of its ability to increase the endogenous production and the secretion of sebum and to strengthen the barrier effect of the skin (U.S. Pat. No. 4,496,556). U.S. Pat. No. 5,843,932 has also disclosed the use of DHEA in overcoming atrophy of the dermis by inhibiting loss of collagen and of connective tissue. In addition, patent U.S. Pat. No. 5,736,537 has disclosed the use by the oral route of DHEA esters, in particular of DHEA salicylate, in regulating atrophy of the skin due to general deterioration or thinning of the dermis. Finally, provision has been made to use DHEA sulfate for treating various signs of aging, such as wrinkles, loss of radiance of the skin and cutaneous slackening (EP-0 723 775).

However, to the knowledge of the Applicant Company, it has never yet been suggested to combine DHEA and/or its precursors or derivatives with an NO-synthase inhibitor.

A subject matter of the invention is thus a composition comprising, in a physiologically acceptable medium, DHEA and/or a chemical or biological precursor or derivative of the latter, in combination with at least one NO-synthase inhibitor.

DHEA has the following formula (I):

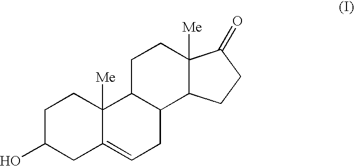

It is, for example, available from Akzo Nobel.

The term “DHEA precursors” is understood to mean its biological precursors which are capable of being converted to DHEA during metabolism and its chemical precursors which can be converted to DHEA by exogenous chemical reaction. Examples of biological precursors are Δ5-pregnenolone, 17α-hydroxypregnenolone and 17α-hydroxypregnenolone sulfate, without this list being limiting. Examples of chemical precursors are sapogenins and their derivatives, such as diosgenin (or spirost-5-en-3β-ol), hecogenin, hecogenin acetate, smilagenin and sarsapogenin, and the natural extracts comprising them, in particular fenugreek and extracts of Dioscoreae, such as wild yam root, without this list being limiting.

The term “DHEA derivatives” is understood to mean both its biological derivatives and its chemical derivatives. Mention may in particular be made, as biological derivatives, of Δ5-androstene-3,17-diol and Δ4-androstene-3,17-dione, without this list being limiting. Mention may in particular be made, as chemical derivatives, of DHEA salts, in particular water-soluble salts, such as DHEA sulfate. Mention may also be made of esters, such as the esters of hydroxycarboxylic acids and of DHEA disclosed in particular in U.S. Pat. No. 5,736,537 or other esters, such as DHEA salicylate, acetate, valerate (or n-heptanoate) and enanthate. Mention may also be made of the DHEA derivatives (DHEA carbamates, DHEA 2-hydroxymalonate esters and DHEA amino acid esters) disclosed in application FR 00/03846 on behalf of the Applicant Company. This list is obviously not limiting.

The biological and chemical precursors and derivatives of DHEA will be denoted below by “DHEA analogs”.

For its part, the NO-synthase inhibitor is chosen from compounds which inhibit the synthesis and/or which accelerate the catabolism of NO-synthase, compounds which neutralize NO-synthase or compounds which are involved in adjusting the signal transduced by NO-synthase. Thus, according to the invention, NO-synthase inhibitors are products which make it possible, in situ in man, to partially, indeed even totally, inhibit the synthesis of nitrogen monoxide (NO).

NO-synthases exist in three forms, namely two constituent forms, combining neuronal NO-synthase (or NOS 1) and endothelial NO-synthase (or NOS 3), and an inducible form (or NOS 2) (Medecine/Sciences, 1992, 8, pp. 843-845). In the present application, the term “NO-synthase” covers all the isoforms of the enzyme.

The NO-synthase inhibitor can be chosen from: N^{G}-monomethyl-L-arginine (NMMA), N^{G}-nitro-L-arginine; the methyl ester of N^{G}-nitro-L-arginine; diphenyleneiodonium chloride; 7-nitroindazole; N(5)-(1-iminoethyl)-L-ornithine; NGN^{G}-dimethyl-L-arginine; NGN^{G}-dimethylarginine; 2-(4-carboxyphenyl)-4,4,5,5-tetramethylimidazoline-1-oxy-3-oxide; aminoguanidine; canavanine and ebselen.

In an alternative form, and according to a preferred embodiment, the NO-synthase inhibitor according to the invention can be chosen from: lipochroman-6, a grape extract, an olive tree extract, a Gingko biloba extract or epicatechin.

Lipochroman-6 is a compound corresponding to the general formula:

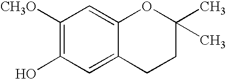

The term “grape extract” is understood to mean a plant extract of the species Vitis vinifera, which is sold in particular by Euromed under the name Leucocyanidines de raisins extra, or by Indena under the name Leucoselect®, or, finally, by Hansen under the name extrait de marc de raisin.

The term “olive tree extract” is understood to mean a plant extract of the species Olea europaea, which is preferably obtained from olive tree leaves. This extract is sold in particular by Vinyals in the form of a dry extract or by Biologia & Technologia under the trade name Eurol BT.

The term “Gingko biloba extract” is understood to mean an extract from a plant of the species Gingko biloba. Preferably, according to the invention, use is made of a dry aqueous extract from this plant sold by Beaufour under the trade name Ginkgo biloba extrait standard.

Epichatechin or 2-[3,4-dihydroxyphenyl]-3,4-dihydro-1[2H]-benzopyran-3,5,7-triol is a natural component of green tea. It can be provided in the form of two enantiomers, namely: (+)-epichatechin or [2S,3S]-2-[3,4-dihydroxyphenyl]-3,4-dihydro-1[2H]-benzopyran-3,5,7-triol; and (−)-epichatechin or [2S,3S]-2-[3,4-dihydroxyphenyl]-3,4-dihydro-1[2H]-benzopyran-3,5,7-triol.

According to the invention, each of these compounds can be used alone. However, the invention also relates to the use of a mixture in any proportion of (+)-epichatechin and of (−)-epichatechin. In addition to these two enantiomeric forms, the invention also relates to the analogs of epichatechin and/or its derivatives, with the exception of epichatechin gallate.

Thus, otherwise in the text and for simplicity, the term “epichatechin” is understood, unless otherwise indicated, as meaning (+)-epichatechin or (−)-epichatechin or a mixture in any proportion of (+)-epichatechin and of (−)-epichatechin, and the analogs of epichatechin and/or its derivatives, with the exception of epichatechin gallate.

The composition according to the invention may be intended for topical application to the skin or hair. In this case, the DHEA and/or its analogs can be present in an amount ranging from 0.0001 to 10% by weight and preferably from 0.001 to 5% by weight, with respect to the total weight of the composition. Better still, the DHEA and/or its analogs can be present in an amount of approximately 1% by weight, with respect to the total weight of the composition. For its part, the NO-synthase inhibitor can represent from 0.001 to 1% of the total weight of the composition.

The composition according to the invention can be provided in any pharmaceutical dosage form normally used in the cosmetics and dermatological fields and it can be in particular in the form of an optionally gelled aqueous solution, of a dispersion, optionally two-phase, of the lotion type, of an emulsion obtained by dispersion of a fatty phase in an aqueous phase (O/W) or vice versa (W/O), or of a triple emulsion (W/O/W or O/W/O), or of a vesicular dispersion of ionic and/or nonionic type. These compositions are prepared according to the usual methods.

This composition can be more or less fluid and can have the appearance of a white or colored cream, of an ointment, of a milk, of a lotion, of a serum, of a paste or of a foam. It can optionally be applied to the skin in the form of an aerosol. It can also be provided in the solid form, in particular in the form of a stick. It can be used as a care product and/or as a make-up product for the skin.

In addition, insofar as NO-synthase inhibitors are already known for inhibiting hair loss (FR 00/05524), the Applicant Company believes, without wishing to be committed to this theory, that they will be capable of reinforcing the beneficial effect of hair compositions based on DHEA or analogs, in which DHEA is itself known to be effective against canities (FR 99/12773).

Consequently, the composition according to the invention can, in an alternative form, be a hair composition which is provided in particular in the form of a shampoo or of a conditioner, for example.

In a known way, the composition of the invention can also comprise the adjuvants used in the cosmetics field, such as hydrophilic or lipophilic gelling agents, hydrophilic or lipophilic active principles, preservatives, antioxidants, solvents, fragrances, fillers, screening agents, pigments, odor absorbers and coloring materials. The amounts of these various adjuvants are those conventionally used in the field under consideration, for example from 0.01 to 20% of the total weight of the composition. These adjuvants, depending upon their nature, can be introduced into the fatty phase, into the aqueous phase, into the lipid vesicles and/or into the nanoparticles. In any event, these adjuvants, and their proportions, will be chosen so as not to harm the properties desired for the combination of active principles according to the invention.

When the composition of the invention is an emulsion, the proportion of the fatty phase can range from 5 to 80% by weight and preferably from 5 to 50% by weight, with respect to the total weight of the composition. The oils, the emulsifiers and the coemulsifiers used in the composition in the form of an emulsion are chosen from those conventionally used in the field under consideration. The emulsifier and the coemulsifier are present in the composition in a proportion ranging from 0.3 to 30% by weight and preferably from 0.5 to 20% by weight, with respect to the total weight of the composition.

Mention may be made, as oils which can be used in the invention, of mineral oils (liquid petrolatum), oils of vegetable origin (avocado oil, soybean oil), oils of animal origin (lanolin), synthetic oils (perhydrosqualene), silicone oils (cyclomethicone) and fluorinated oils (perfluoropolyethers). Use may also be made, as fatty substances, of fatty alcohols (cetyl alcohol), fatty acids or waxes (carnauba wax, ozokerite).

Mention may be made, as emulsifiers and coemulsifiers which can be used in the invention, of, for example, esters of fatty acid and of polyethylene glycol, such as PEG-20 stearate, and esters of fatty acid and of glycerol, such as glyceryl stearate.

Mention may in particular be made, as hydrophilic gelling agents, of carboxyvinyl polymers (carbomer), acrylic copolymers, such as acrylate/alkyl acrylate copolymers, polyacrylamides, polysaccharides, natural gums and clays, and mention may be made, as lipophilic gelling agents, of modified clays, such as bentones, metal salts of fatty acids, hydrophobic silica and polyethylenes.

Use may in particular be made, as active principles, of depigmenting agents and keratolytic and/or desquamating agents.

In the event of incompatibility, the active principles indicated above and/or the DHEA analogs according to the invention can be incorporated in spherules, in particular ionic or nonionic vesicles and/or nanoparticles (nanocapsules and/or nanospheres), so as to isolate them from one another in the composition.

According to another possibility, the composition according to the invention may be intended for administration by the oral route.

In this case, it can be provided in any pharmaceutical dosage form suitable for this method of administration, for example in the form of divisible or nondivisible tablets, of granules, of capsules, including gelatin capsules, of medicinal solutions, of suspensions or of solutions comprising an appropriate excipient.

The daily doses of DHEA or analogs administered by the oral route can be between 1 and 100 mg/day, preferably between 25 and 75 mg/day. Preferably, the DHEA or analog is present in the composition according to the invention in an amount which makes possible its administration at a dose of between 50 and 100 mg/day, said dosage being achieved with a composition taken one or more times, with a unit dose of 50 mg.

Whatever its method of administration, the composition according to the invention can be used to prevent and/or treat irritation of the skin and/or sensitive skin and/or signs of cutaneous aging.

The term “irritation of the skin” is understood to mean any form of irritation resulting from the application to the skin of chemicals of natural or synthetic origin, for example used in cosmetics or dermatology, and which may be reflected in particular by red blotches, pain or tingling.

The expression “sensitive skin” covers both irritable and/or reactive skin and intolerant skin.

An irritable and/or reactive skin is a skin which reacts with pruritus, that is to say with itching or with tingling, to various factors, such as the environment, emotions, food, wind, rubbing, shaving, soap, surfactants, hard water with a high concentration of calcium, temperature variations or wool. In general, these signs are associated with a dry skin, with or without dry patches, or with a skin exhibiting erythema.

An intolerant skin is a skin which reacts with feelings of warming, of tightness, of pins and needles and/or of red blotches to various factors, such as the environment, emotions, food and some cosmetics. In general, these signs are associated with a hyper-seborrheic or acneic skin, with or without dry patches, and with erythema.

Finally, the term “signs of cutaneous aging” is understood to mean any change in the external appearance of the skin due to aging, whether chrono-biological and/or photoinduced, such as, for example, wrinkles and fine lines, a withered skin, a soft skin, a thinned skin, or lack of elasticity and/or of tone of the skin.

The present invention thus also relates to a cosmetic process for preventing and/or treating irritation of the skin and/or sensitive skin and/or signs of cutaneous aging, comprising the administration, by the topical or oral route, of a composition comprising DHEA and/or a chemical or biological precursor or derivative of the latter, in combination with at least one NO-synthase inhibitor.

In an alternative form, the composition according to the invention can be used for the treatment of the hair and/or scalp, in particular for preventing and/or treating canities and/or hair loss.

The present invention thus also relates to a cosmetic process for the treatment of the hair and/or scalp comprising the administration, by the topical or oral route, of the composition according to the invention.

According to yet another possibility, the composition according to the invention can be used as reducing composition. This is because DHEA is known for its inhibiting effect on the differentiation of adipocytes, while NO-synthase inhibitors have already been described as capable of acting as stimulants of lipolysis. The combination of these two types of active principles thus makes it possible to reinforce their effects for the purpose of rendering the figure more visibly slimmer.

The invention will be better understood, and its advantages will emerge more clearly, in the light of the following examples, which are given by way of illustration and without implied limitation. In these examples, the amounts are shown as percentage by weight.

### EXAMPLES

### Example 1

### Composition for Oral Administration

Soft capsules are prepared in a way conventional to a person skilled in the art, these capsules having the following composition:

| | | |
|---|---|---|
| Hydrogenated soybean oil | . . . | 40 mg |
| Wheat oil | . . . | 95 mg |
| Soybean lecithin | . . . | 20 mg |
| Natural tocopherols | . . . | 5 mg |
| Ascorbic acid | . . . | 30 mg |
| Encapsulated dry extract of Vitis vinifera | . . . | 300 mg |
| sold by Indena under the trade name | | |
| “Phytosome de Leucoselect”) | | |
| (corresponding to approximately 100 mg of | | |
| extract of Vitis vinifera) | | |
| DHEA | . . . | 50 mg |

### Example 2

### Composition for Topical Application

A care cream (oil-in-water emulsion) is prepared in a way conventional to a person skilled in the art, this cream having the following composition: Dry aqueous extract of Gingko biloba, sold by Beaufour under the trade name “Gingko biloba

| | | |
|---|---|---|
| extrait standard” | . . . | 5% |
| DREA | . . . | 0.1% |
| Glyceryl stearate | . . . | 2% |
| Polysorbate 60 (Tween 60 ®, sold by ICI) | . . . | 1% |
| Stearic acid | . . . | 1.4% |
| Triethanolamine | . . . | 0.7% |
| Carbomer | . . . | 0.4% |
| Liquid fraction of karite butter | . . . | 12% |
| Perhydrosqualene | . . . | 12% |
| Fragrance | . . . | 0.5% |
| Preservative | . . . | q.s. |
| Water | . . . | q.s. for 100% |

1. A composition comprising, in a physiologically acceptable medium, a sapogenin or a natural extract comprising it, in combination with at least one NO-synthase inhibitor.

2. The composition as claimed in claim 1, characterized in that said sapogenin is chosen from diosgenin, hecogenin, smilagenin and sarsapogenin.

3. The composition as claimed in claim 1, characterized in that said natural extract is chosen from fenugreek and extracts of Dioscorea.

4. The composition as claimed in claim 3, characterized in that said extract of Dioscorea is a wild yam root extract.

5. The composition as claimed in any one of the preceding claims, characterized in that the NO-synthase inhibitor is chosen from: lipochroman-6, a grape extract, an olive tree extract, a Gingko biloba extract or epicatechin.

6. The composition as claimed in any one of the preceding claims, characterized in that it is intended for topical application to the skin.

7. The composition as claimed in claim 6, characterized in that the sapogenin is present in an amount ranging from 0.0001 to 10% by weight, with respect to the total weight of the composition.

8. The composition as claimed in claim 7, characterized in that the sapogenin is present in an amount ranging from 0.001 to 5% by weight, with respect to the total weight of the composition.

9. The composition as claimed in claim 8, characterized in that the sapogenin is present in an amount of approximately 1% by weight, with respect to the total weight of the composition.

10. The composition as claimed in any one of claims 6 to 9, characterized in that the NO-synthase inhibitor represents from 0.001 to 1% of the total weight of the composition.

11. The composition as claimed in any one of claims 1 to 5, characterized in that it is intended for administration by the oral route.

12. A cosmetic process for preventing and/or treating irritation of the skin and/or sensitive skin and/or signs of cutaneous aging, comprising the administration, by the topical or oral route, of a composition comprising DHEA and/or a chemical or biological precursor or derivative of the latter, in combination with at least one NO-synthase inhibitor.

13. The process as claimed in claim 12, characterized in that DHEA or its precursor or derivative is administered by the oral route in a proportion of 1 to 100 mg/day, preferably of 25 to 75 mg/day.

14. A cosmetic process for treating the hair and/or scalp, comprising the administration, by the topical or oral route, of a composition comprising DHEA and/or a chemical or biological precursor or derivative of the latter, in combination with at least one NO-synthase inhibitor.

15. The process as claimed in any one of claims 12 to 14, characterized in that said chemical derivative is chosen from DHEA salts and esters.

16. The process as claimed in any one of claim 12 to 14, characterized in that said chemical precursor is chosen from sapogenins, their derivatives, and natural extracts comprising them.

17. Cosmetic use of the composition as claimed in any one of claims 1 to 11 as reducing composition.
